(19) United States
(12) Patent Application Publication
(10) Pub. No.: US 2004/0014165 A1
(43) Pub. Date: Jan. 22, 2004

(54) System and automated and remote histological analysis and new drug assessment

(76) Inventors: Joseph Keidar, Moshav Yokneam (IL); Nir Karasikov, Haifa (IL); Alfred Brukstein, Haifa (IL); Gal Goshen, Safed (IL); Jona Bela, Jerusalem (IL)

Correspondence Address: Edward Langer, Landon & Stark Associates, Crystal Park One Suite 210, 2011 Crystal Drive, Arlington, VA, 22202-3709, US

(21) Appl. No.: 10/311,430

(22) PCT Filed: Jun. 19, 2001
(86) PCT No.: PCT/IL01/00561

(30) Foreign Application Priority Data
Jun. 19, 2000 (IL) 136884

(51) Int. Cl. G06F 19/00; G01N 33/48; G01N 33/50; G01N 1/30

(52) U.S. Cl. 435/40.5; 702/19

(57) ABSTRACT

A histological system includes: a database, comprising histological images and quantitative information regarding diagnostically relevant features of histological samples; an inspection procedure for the examination of a suspected object; and analysis tools for comparison of the locally obtained data to those stored in the database and for applying classification algorithms based on heuristically derived histological diagnoses.

### FIELD OF THE INVENTION

The present invention relates to the diagnosis and peridiagnosis of histological tissues. More particularly, the present invention discloses a method and system useful for improving and standardizing cancer detection and grading in an automated or semi-automated way and which facilitates transfer of acquired images for remote diagnosis and teleconsultation. Additionally, the present invention is useful as a system for automatic or remote assessment of new drug evaluation assays.

### BACKGROUND OF THE INVENTION

It is widely accepted that the need for histological analysis is part of the routine procedure for cancer detection. Cancer is a lethal disease that kills millions of patients every year. The diagnosis of cancer is based principally on techniques that have been accepted for a relatively long time. The classic diagnosis of most neoplastic diseases is carried out by means of a number of histological examinations of suspect tissues. Subsequently, for some types of cancer and for some cases in which the diagnosis is particularly difficult, other diagnostic methods are used, which are based on immunological methods comprising specific antibodies and special stains.

The presently used techniques are limited in that they are based on qualitative diagnosis, which can be highly subjective according to the experience of the pathologist. A more objective quantitative approach used for automated diagnosis has not been developed heretofore. Presently, the diagnosis procedure almost always requires performing a biopsy, in order to establish the nature of the lesion, before any treatment involving the surgical removal of the cancerous tissue, chemotherapy, etc. Thus, pathological histology is accepted as the main means for the diagnosis of cancer. Previous developments in automatic and remote tissue sample diagnosis were all addressed to cytological diagnosis, including for example products for Pap Smear and others that have been introduced, whereas scarce work has been conducted for automation of histological diagnosis.

Additionally, it is clear from the references which follow that a brute-force approach using many features at a time does not necessarily help; it can blur the separation between normal and malignant tissues.

The references include:

M. Kearns and U. Vazirani, “An introduction to the Computational Learning Theory”, MIT Press, 1994;

M. Anthony and M. Biggs, “Computational Learning theory”, Cambridge University Press, 1992;

L., G. Valiant “A theory of the learnable”. Communications of ACM, 27(11), 1134-11422, 1984;

A. Blumer, A Ehrenfeucht, D. Haussler and M. Warmuth, “Leamability and the Vapnik-Cheronenkis dimension”, Journal of the ACM, (4), 929-965, 1989;

“A Note on VC-Dimension and Measure of Sets of Reals” by S. Ben-David & L. Gurvits; and

“Combinatorial Variability of Vapnik-Chervonenkis Classes” by S. Ben-David & A. Litman.

### OBJECTS AND SUMMARY OF THE INVENTION

It is a purpose of this invention to provide an improved method for the performance of histological analysis, particularly in respect to the diagnosis of cancer.

It is another purpose of this invention to provide a histological analysis system, which is operable, at least partially, in an automatic manner.

It is a further purpose of this invention to improve diagnostic procedures, to dramatically reduce the danger of false positive or false negative diagnoses.

It is a still further purpose of the invention to offer a new quantitative approach to automate the diagnosis process, presently carried out by pathologists, by use of advanced methods supported by Al software tools.

It is a still further purpose of the invention to provide the pathologist with guidance in this process and to offer a friendlier operating environment to expert pathologists

It is a still further purpose of the invention to apply to the diagnosis process the full advantage of modern communication means and advanced video HW to allow telediagnosis and teleconsultation between experts in severe or marginal cases.

These and other objects not mentioned hereinabove are accomplished by this invention, which provides an extendable database comprising diagnostically relevant features of images and quantitative information (meta-data) supporting them. Typically, the diagnostically relevant features are textural, morphometric, calorimetric and densitometric in nature.

Preferably, the quantitative information is based on a structure and program that are designed so as to provide to the extendable database completeness, accessibility, and as far as possible, universal value.

Optionally, a web site related to the present invention is constructed, comprising a database in accordance with the present invention, system support, pathology information and pathology consultation means.

The invention further comprises an inspection procedure for the examination of a suspected tissue using a motorized stage.

The invention further comprises an embodiment wherein the extendable database is local to the acquisition of data, and where the diagnosis is to be carried out. The local acquisition of data is preferably based on a structure and programming, described further hereinbelow, which correspond to those of the database, so as to facilitate, and preferably to permit, insofar as possible, comparison, feature by feature, of the data obtained locally, representing a specific patient, to those stored in the database.

The database features to be compared are selected by the following procedure:

1) Defining pathologists conclusions regarding an image by a number of features;

2) Reducing said features to mathematical formulas;

3) Using algorithms based on the mathematical formulas to compare numerical data obtained locally with the data comprising the system's database; and

4) refining the conclusions by using artificial intelligence tools.

By means of image capturing devices, at least six images are acquired: one image, of normal tissue near its border with suspected tissue, two images of the boundary between the suspected tissue, and normal tissue, two more of the suspected tissue only, and one image in lower resolution of the entire suspected area. Then the images undergo a standardization procedure and object features as well as textural features are extracted. The features are compared to the data comprising the database. The comparison results are not just a binary decision but a graded scoring (for example between 0 and 1) that measures the “probability” or grade of malignancy. The comparison is accomplished by means of such artificial intelligence models as the SVM (support-vector machine) technology, as is described in detail in the publication “A Tutorial on Support Vector Machines for Pattern Recognition”, Christopher J. C. Burges, Data Mining and Knowledge Discovery 2, 121-167, 1998, the disclosure of which is entirely incorporated herein by reference for its teaching as to the state of the art on how to use and construct an SVM.

The invention further comprises what may be called aperidiagnosis system, which includes the transfer of images of relevant tissues over the Internet or telephone lines for examinations by experts at locations different from that at which the diagnosis is to be carried out. This transfer via the Internet can be with or without the diagnostic results extracted by the system. The transfer procedure requires validation of the received images in order to insure that the images don't suffer from significant distortion, which can have a destructive effect on image examination.

From the apparatus viewpoint, the diagnosis system of the invention comprises, as components, a standard microscope used in pathology applications, computers, image capturing devices (from microscopes), and telecommunications means. The apparatus components are supplemented, and their operation is improved and controlled by specific diagnosis software.

### BRIEF DESCRIPTION OF DRAWINGS

FIGS. 1A and 1B are photomicrographic images of normal and cancerous tissue respectively and FIG. 1C is an enlarged detail of FIG. 1;

FIG. 2 is a diagram comparing measurement of shape factor values of the normal and cancerous tissues shown in FIGS. 1a-c;

FIG. 3 is a flowsheet schematically illustrating the analysis of pathology samples, according to an embodiment of the invention;

FIG. 4 is a flowsheet illustrating the operation of data preparation, which is part of FIG. 3;

FIG. 5 is a schematic block diagram illustrating an embodiment of Web Site of the invention;

FIG. 6 is a flowsheet illustrating the pathology consultations using said Web Site;

FIG. 7 illustrates a Petri dish carrying out an NDA procedure, in accordance with an exemplary embodiment of the present invention;

FIG. 8. illustrates a sample results graph created by the software of the present invention displaying imaging results derived from Petri dishes used for carrying out a new drug assessment, in accordance with another exemplary embodiment of the present invention;

FIGS. 9a and 9b are tissue samples showing normal and malignant tissue histological sample images, respectively;

FIG. 10a and 10b are tissue histological sample images of malignant and normal tissue showing differences in the complex parameter Heterogeneity and Density;

FIG. 11 is a flow sheet showing the flow of image acquisition and image analysis in an exemplary embodiment of the present invention;

FIGS. 12 (1-7) and (8-16) is Table 1 comprising data values for new features and complex features on Normal Tissue and old features and complex features on Normal Tissue;

FIGS. 13 (1-7) is Table 2 comprising data values for features and complex features on Malignant Tissue;

FIG. 14 is bar graphs showing the features from Tables 1 and 2;

FIG. 15 are Best Couples graphs showing couplings of the data of features and complex features from Tables 1 and 2;

FIG. 16 are Scatter Graphs of data for features from Tables 1 and 2 combined for input into the SVM;

FIG. 17 are Scatter Graphs of data for features from Tables 1 and 2;

FIGS. 18a-c are slides showing a sample as a raw image, enhanced and flattened, respectively; and

FIG. 19 is an example of an SVM plot based on two complex features Best Heterogeneity versus Density.

### DETAILED DESCRIPTION OF PREFERRED EMBODIMENTS

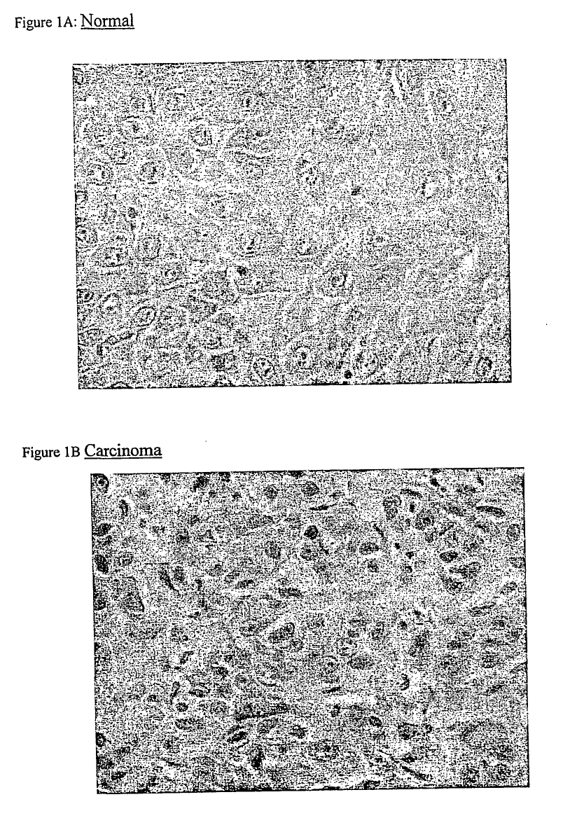

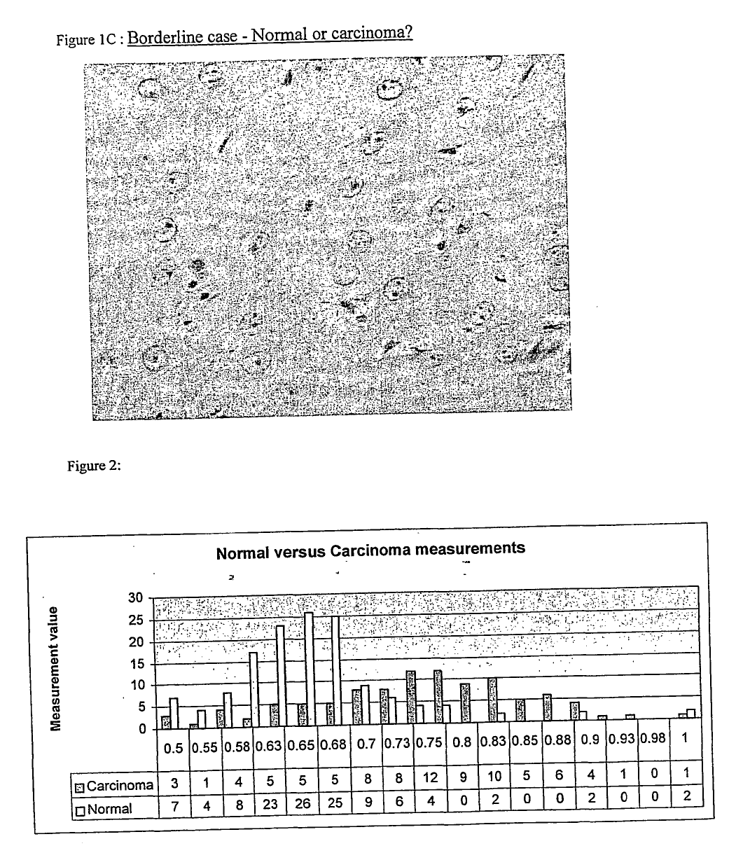

FIG. 1A shows a photomicrographic image of a normal tissue. FIG. 1B shows a comparable image of a cancerous tissue. The images were obtained using regular biological optic magnification of 40× and color video camera with resolution of 768×572 pixels. FIG. 1C shows a borderline case in which it is with difficulty that a firm diagnosis can be made.

FIG. 2 compares normal and carcinoma measurement values of one parameter (Shape Factor). The values indicated by blank rectangles are the normal ones and those indicated by black rectangles are the carcinoma ones. The Statistics results are: Normal Average 0.79 Std., (Standard Deviation 0.036)—Carcinoma Average 0.83 Std (Standard Deviation 0.053), i.e. a large area of overlap and hence difficulty in diagnosis

Whereas the feature demonstrated above only partially separates malignant and normal tissue, the use of multiple features supported by powerful Al techniques yields a high level of reliability in the detection of cancer.

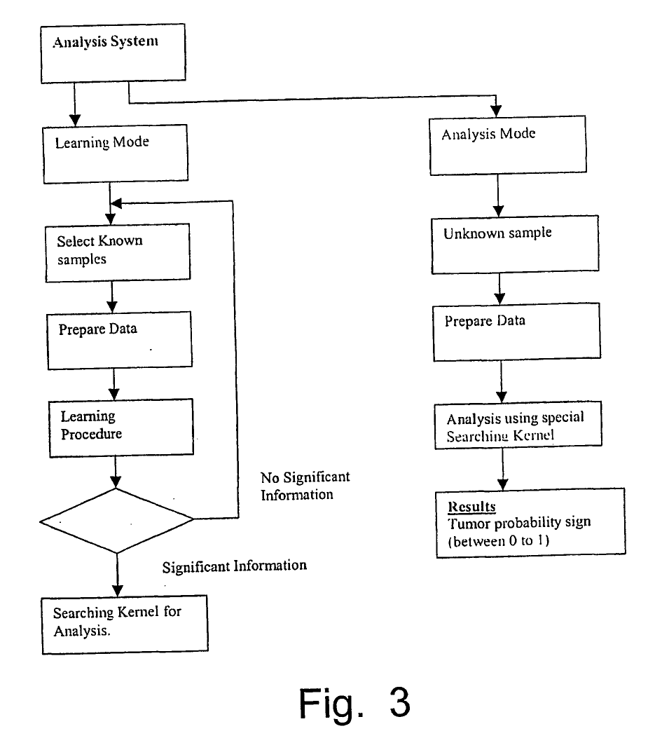

The flow sheet of FIG. 3 illustrates the system of analysis of pathology samples. Since there are different types of cancerous phenomena, which differ as to their character, form and location, in order to diagnose a cancerous growth it is necessary to provide a search kernel that is optimal for each kind of growth. Search kernels are arrays of data, which are expanded and updated frequently. The search kernel for a particular tissue type and patient parameter which will serve as the basis for initial diagnoses, and which will be refined bas diagnoses are added to the extendable database by the SVM as, is initially defined by taking an actual heuristic diagnostic decision from an expert pathologist and translation of the pathologist's input into mathematical algorithms. However, since it is clear that some intangible quality, call it experience or gut-feeling, colors a pathologist's decision-making process, the present invention seeks to employ Al techniques, for example SVM, to fill in the gaps between the pathologists decision and the resulting mathematical formuka and algorithms derived therefrom. SVM is also used to enrich the kernel of features by trying to detect patterns between features previously thought to be unrelated by pathologists, which may have been too subtle for human's to be consciously aware of.

Thus, the system comprises a Learning Mode in which samples of tissues that were already manually diagnosed are analyzed in order to build the search kernel, which is a subset of the features vector or morphometric and/or pathologic features which distinguish cell-types and malignancy types for any particular pathology, and an Analysis Mode in which the search kernel is applied and subsequently refined. In the Learning Mode no new samples are selected; the pertinent data are prepared and the learning procedure is followed. If significant information is thus acquired, the search kernel is searched for analysis in order to update the system. If no significant information is obtained, the procedure returns to the stage prior to the selection of known samples.

In the Analysis Mode, an unknown sample is analyzed. The pertinent data are prepared as will be described hereinbelow and an analysis is carried out by using the search kernels derived from a prior learning system. The results are attributed either by a tumor probability sign, which conveniently is between 0 and 1 or by a sign representing the tumor degree.

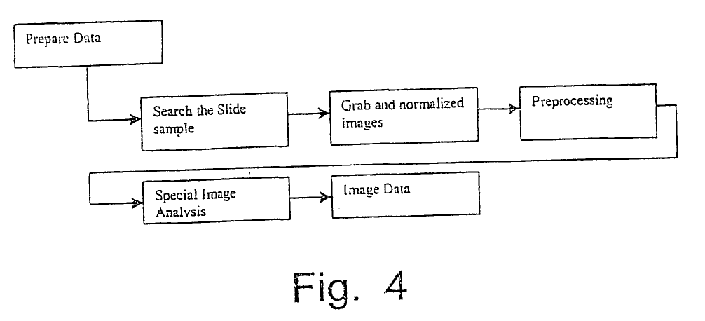

The preparation of data is illustrated in FIG. 4. It comprises the steps of searching the slide samples, obtaining the grab and normalized images, normalization of the selected image (i.e. adjusting light intensity, contrast, etc.), image enhancement, carrying out a special image analysis, and obtaining the image data, which then undergoes local individualizing analysis to adjust for normal variances within a patient population.

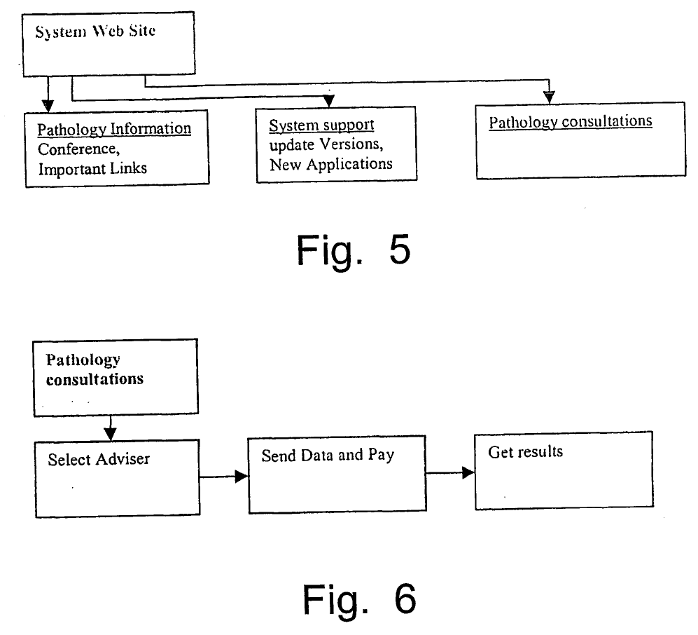

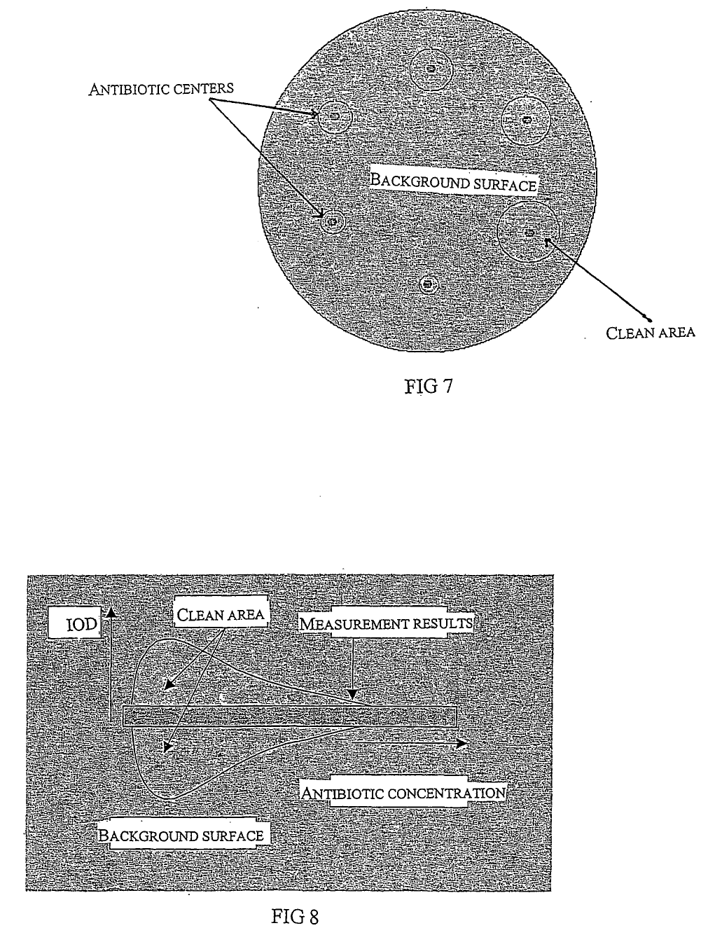

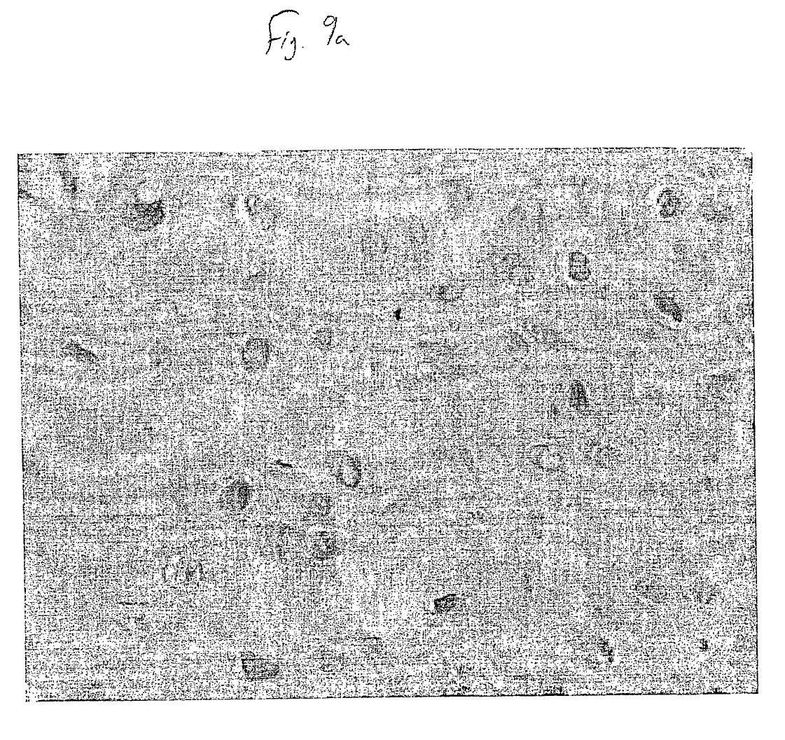

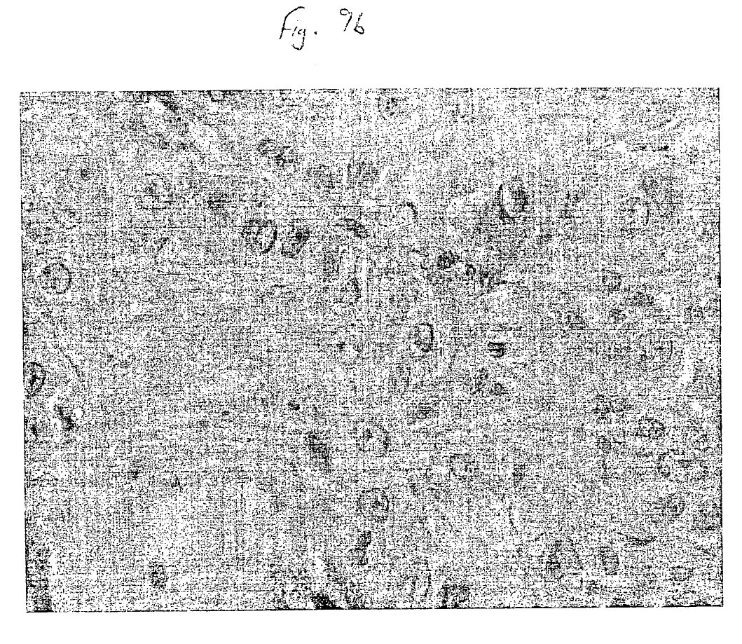

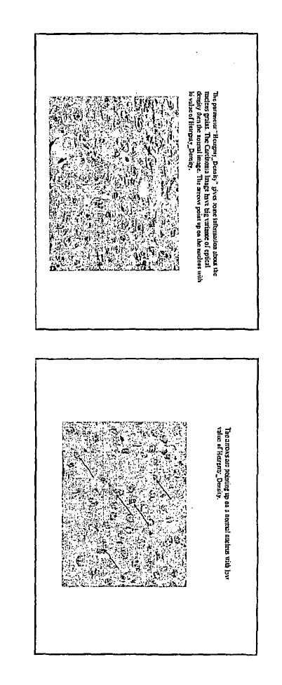

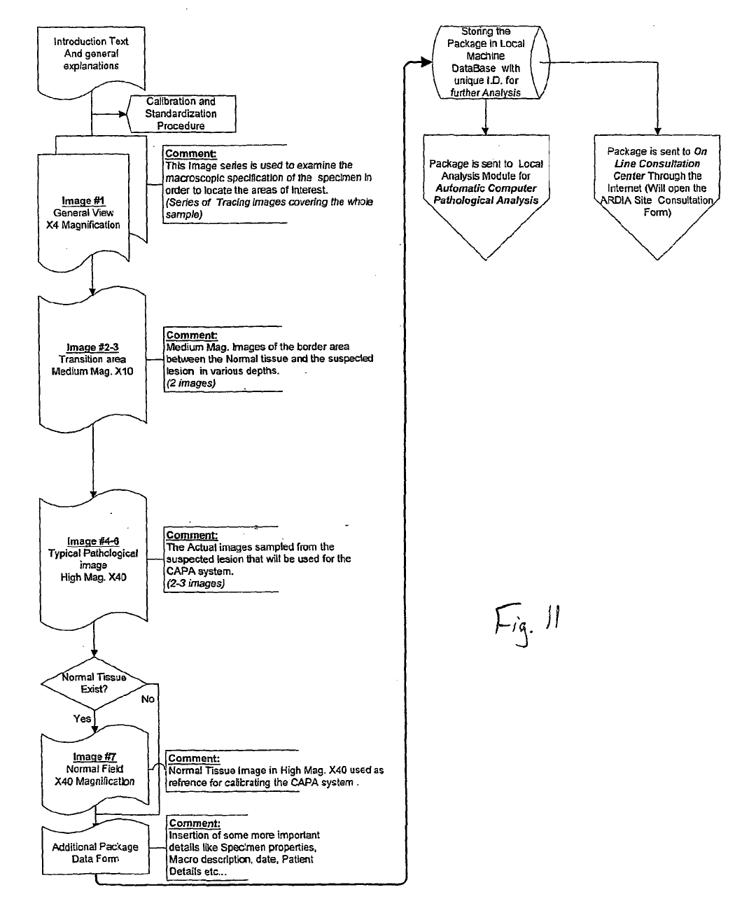

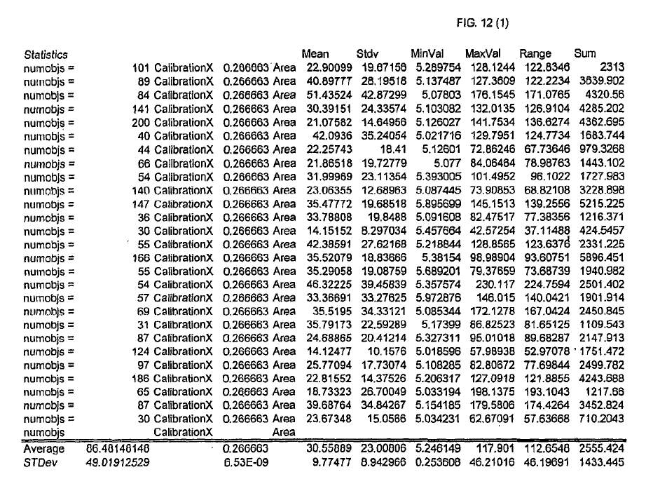

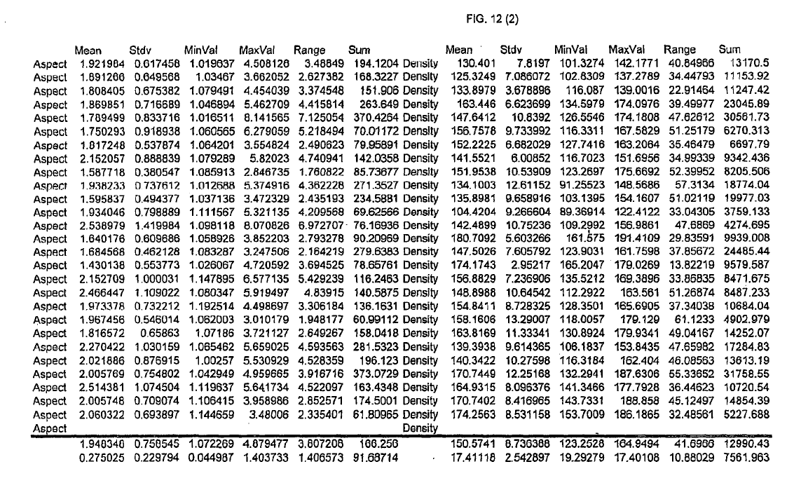

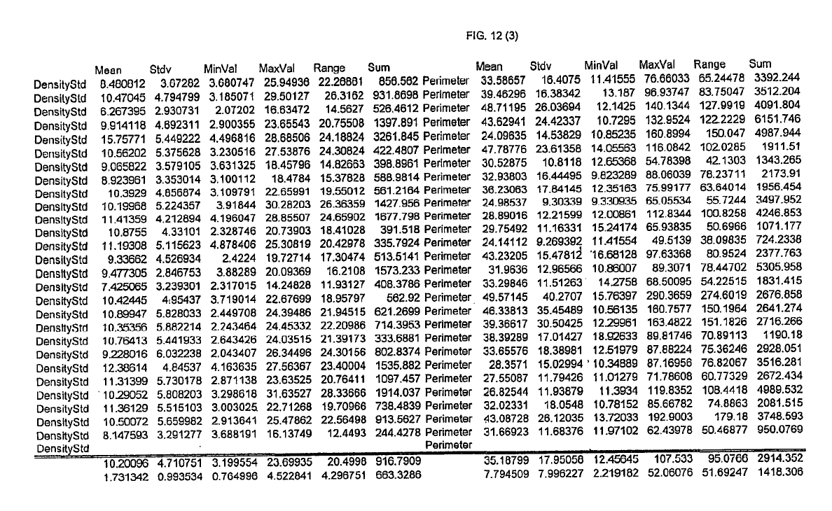

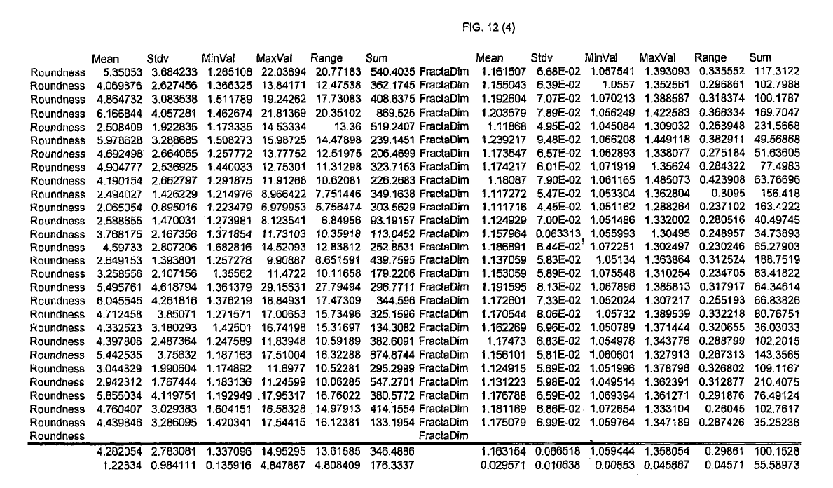

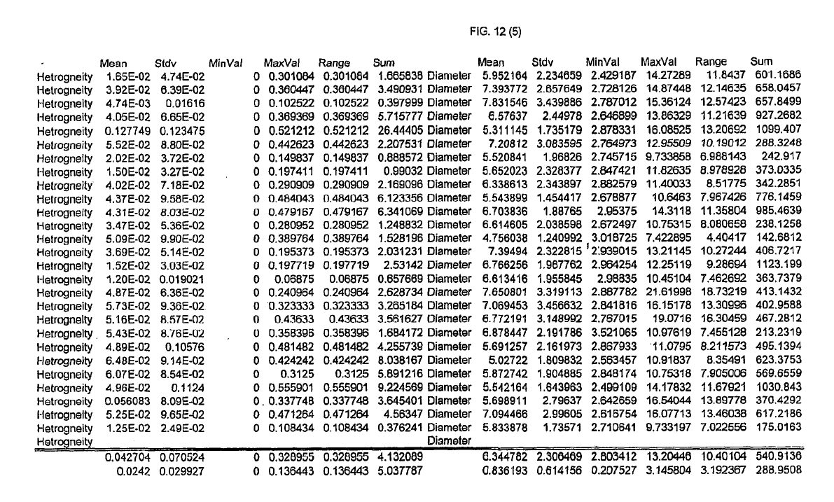

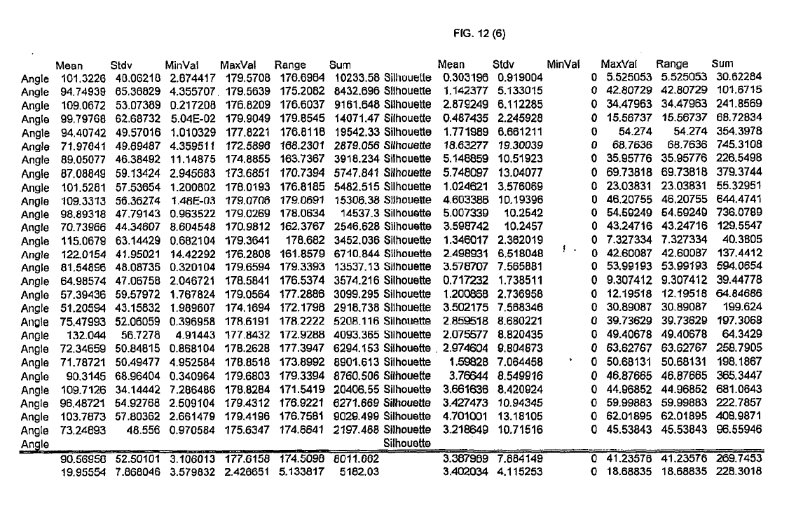

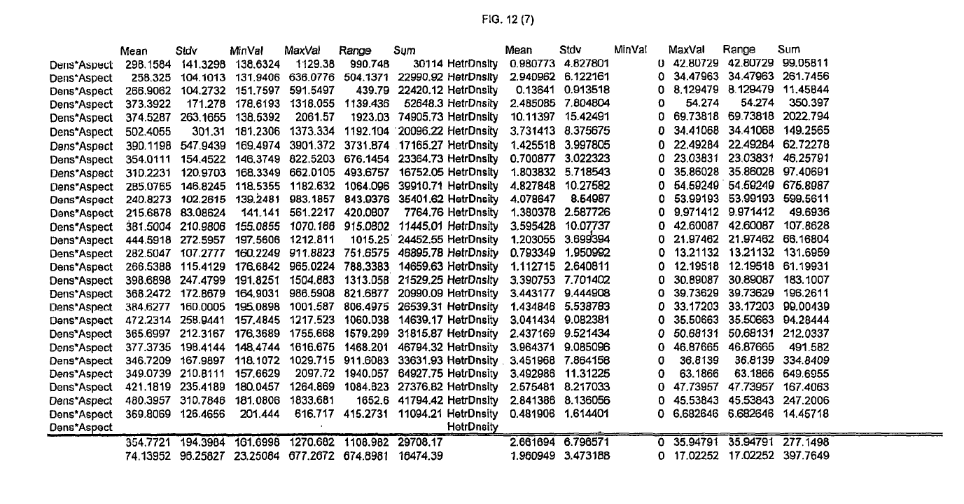

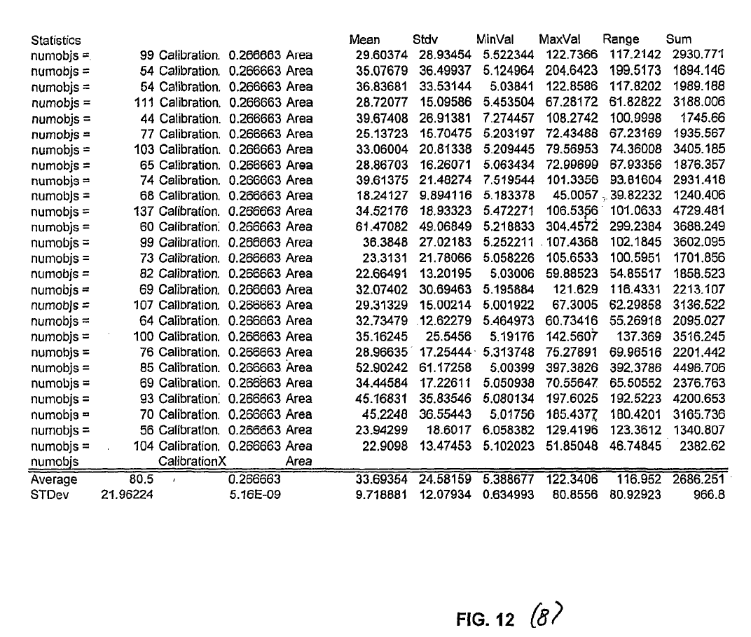

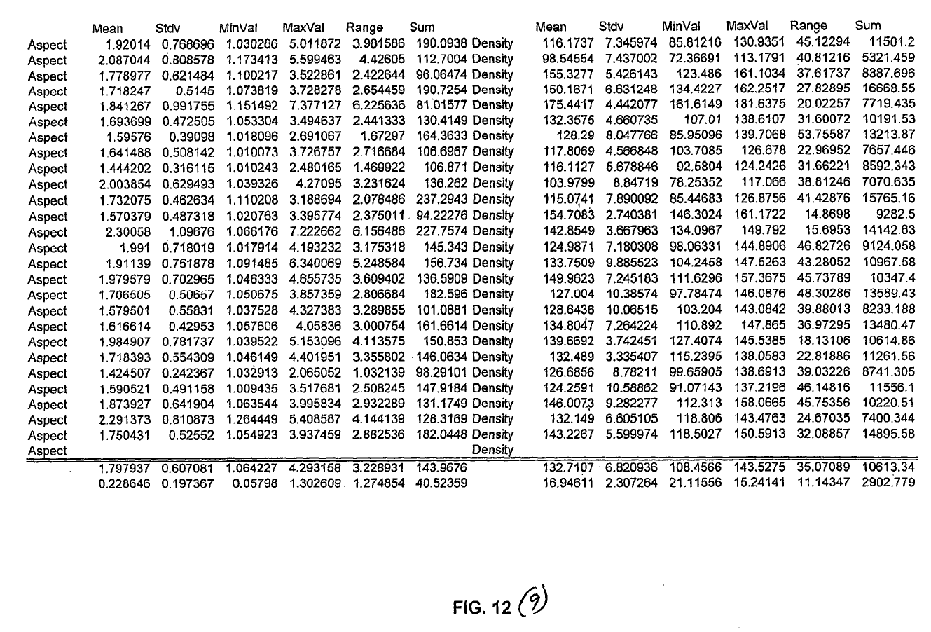

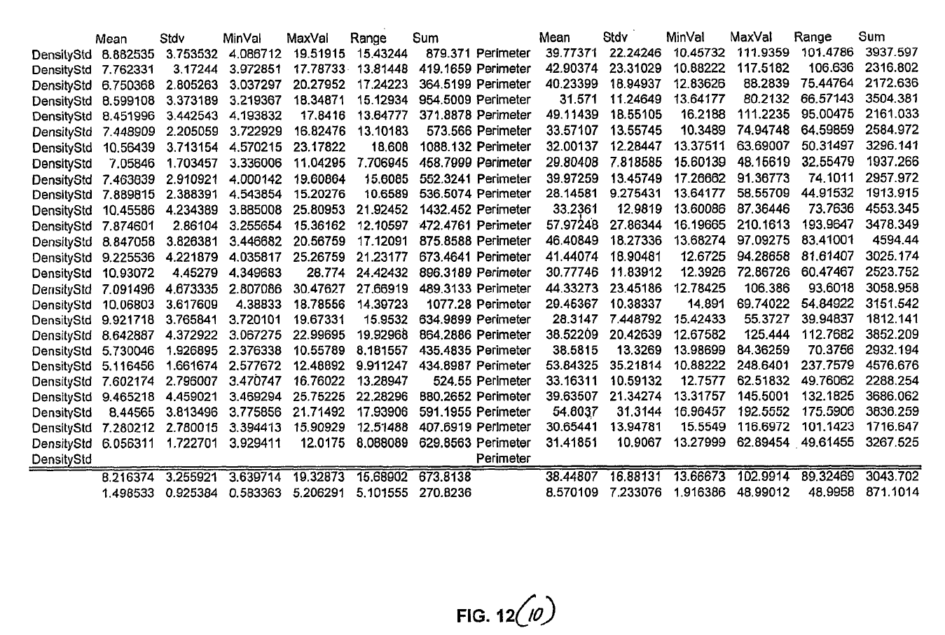

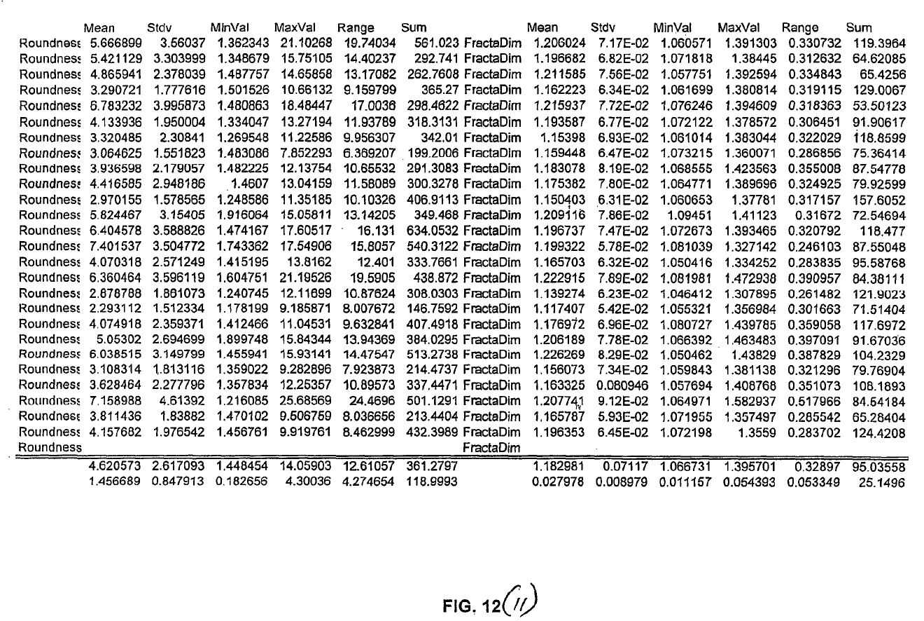

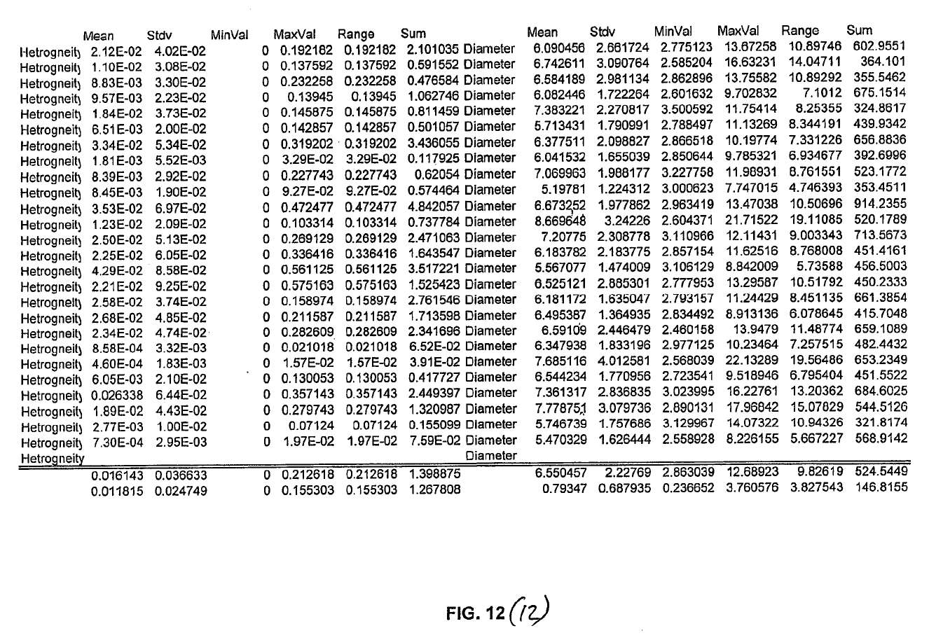

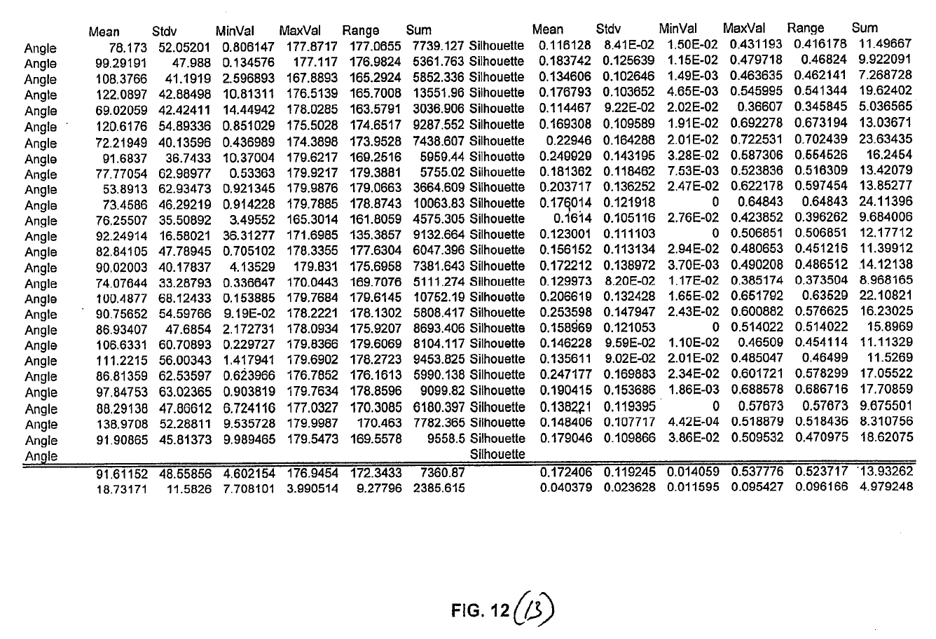

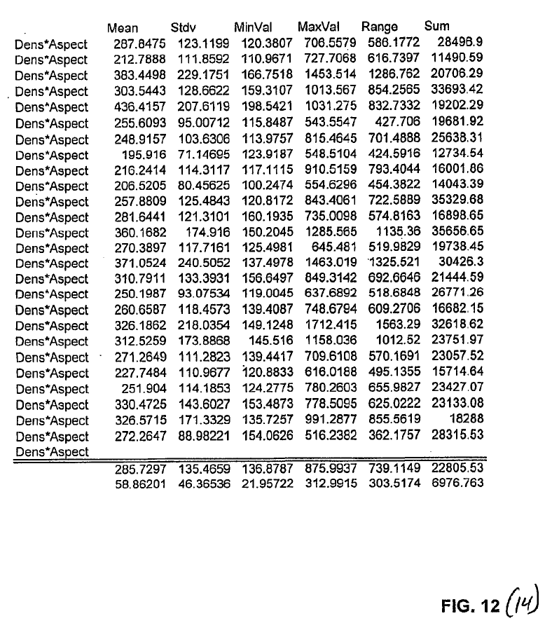

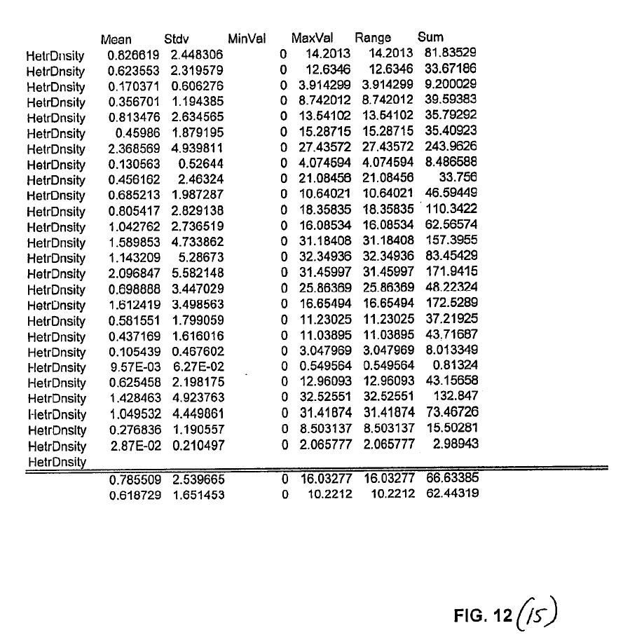

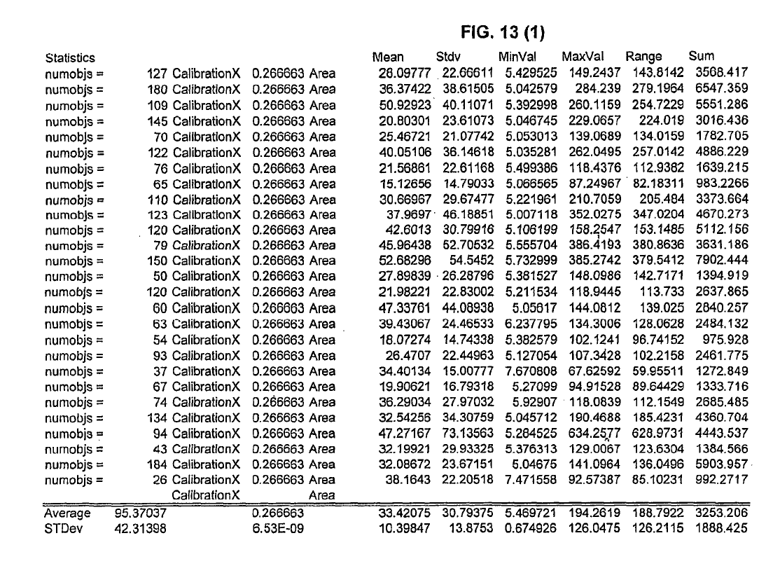

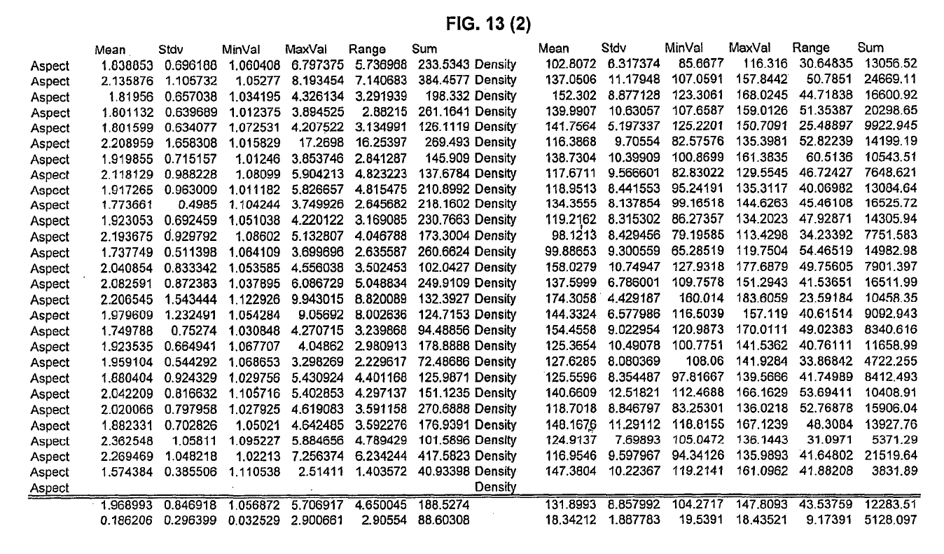

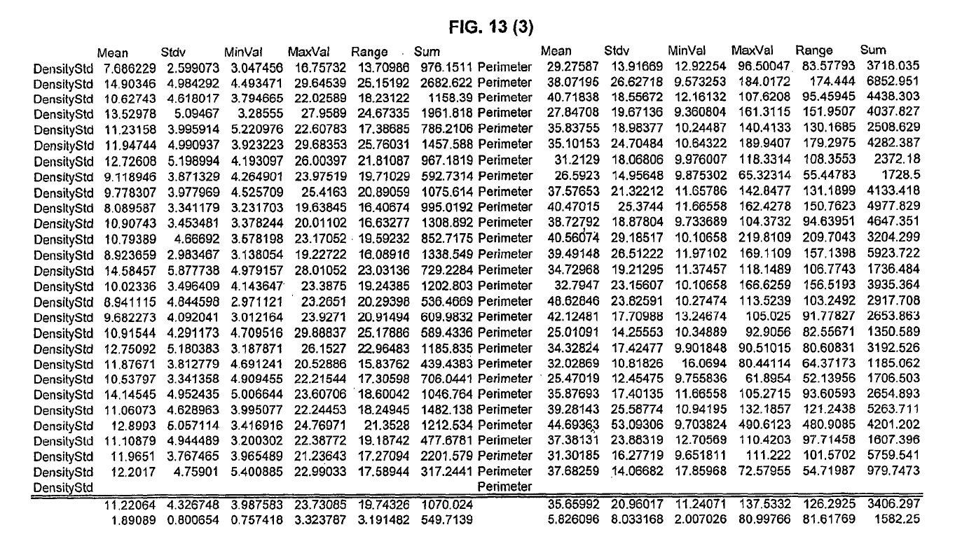

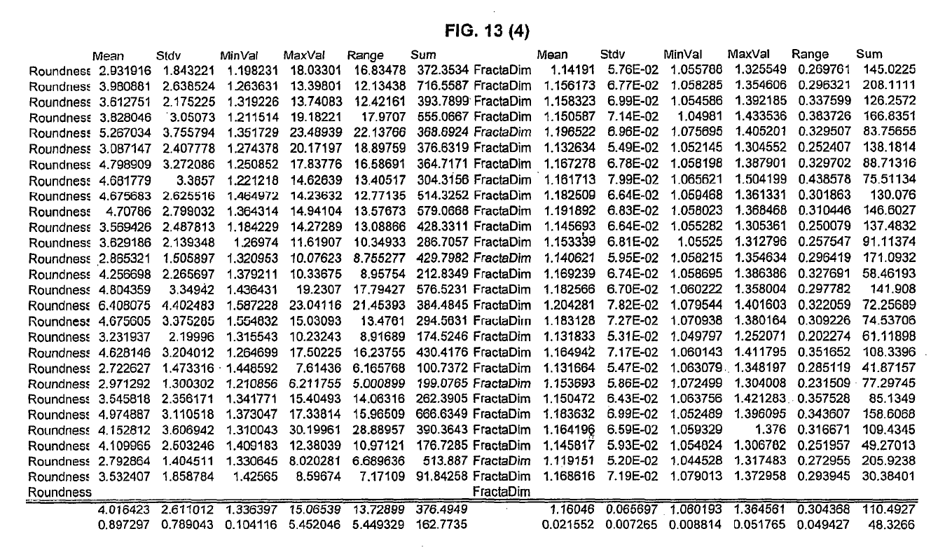

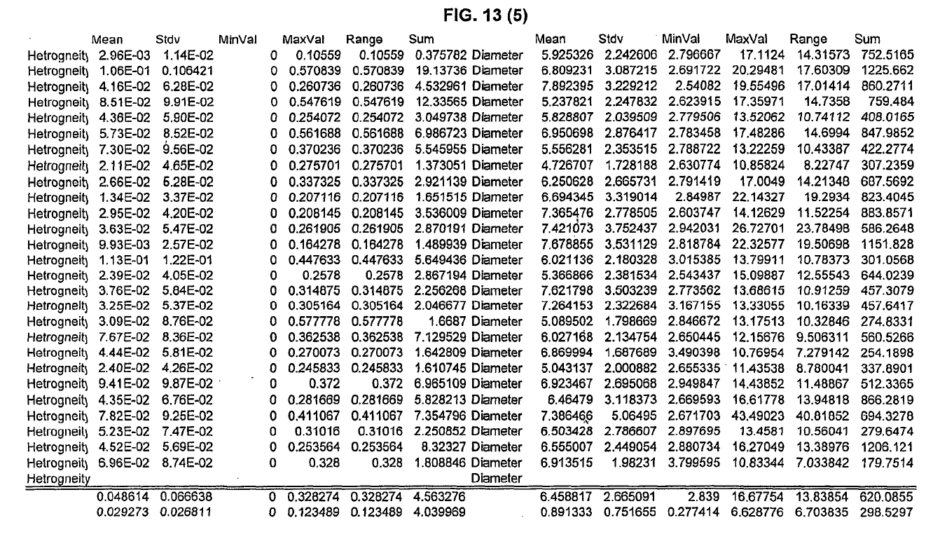

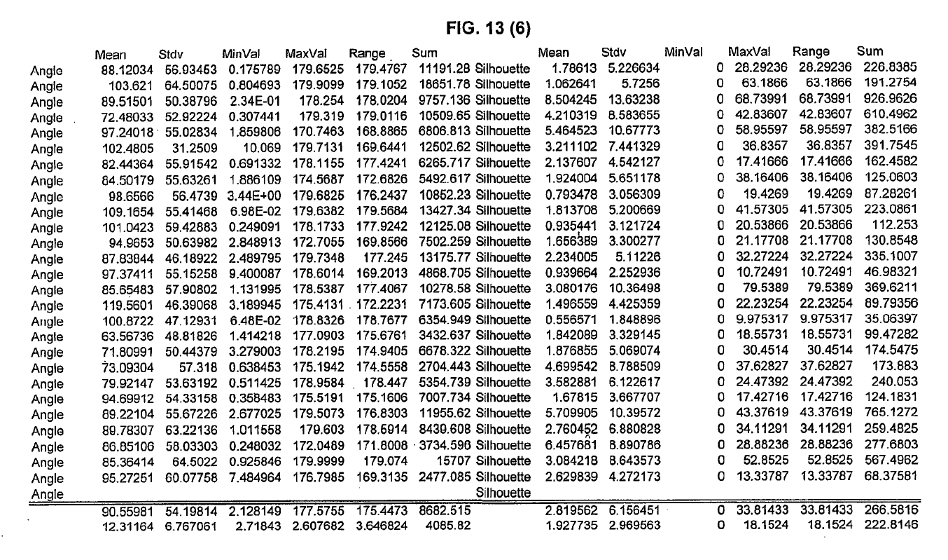

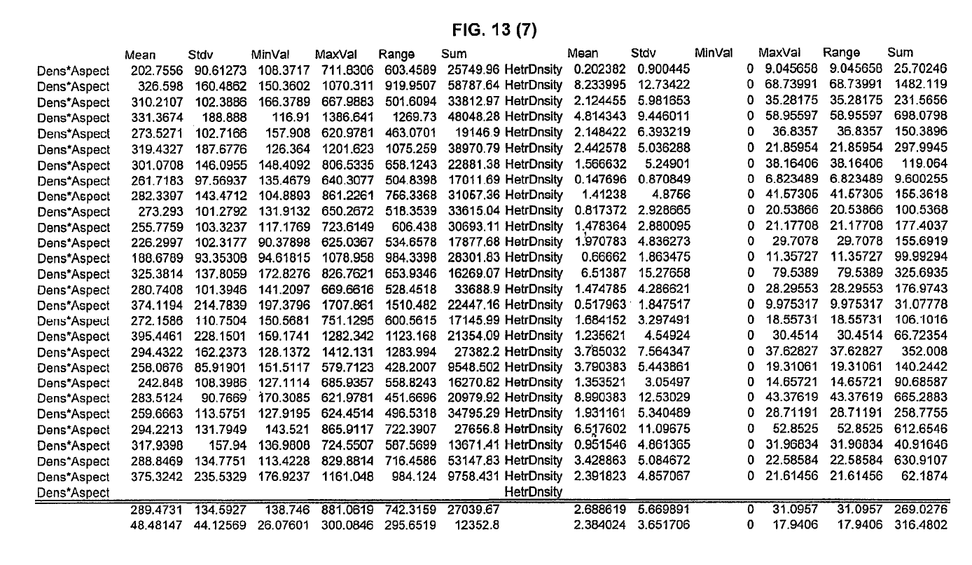

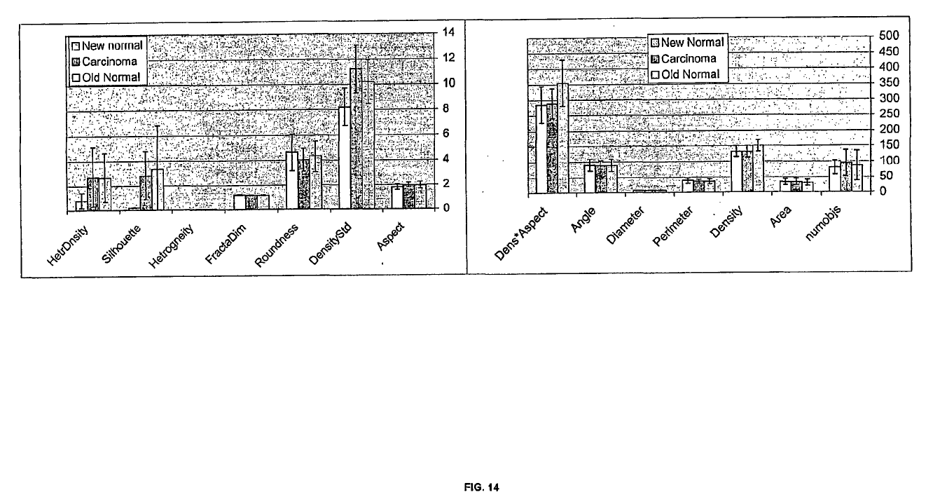

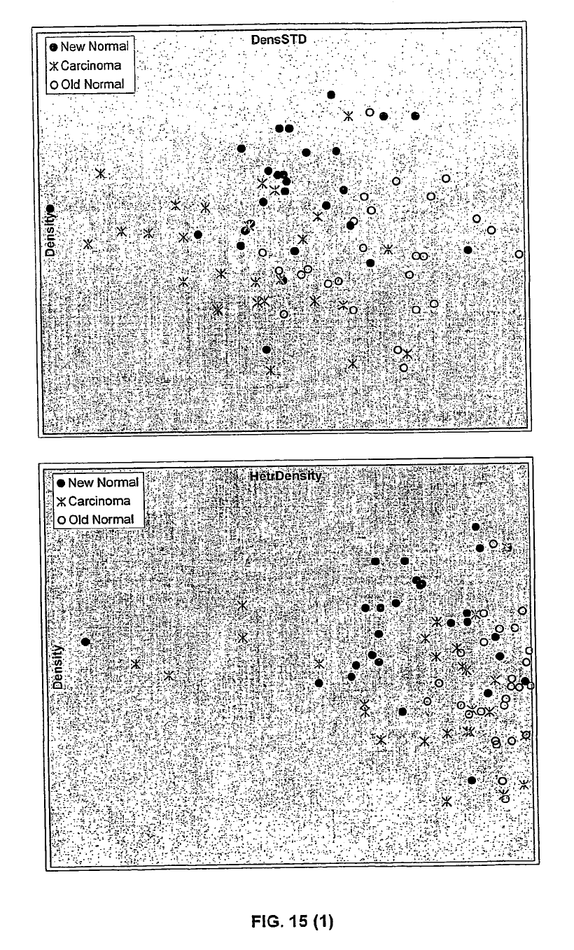

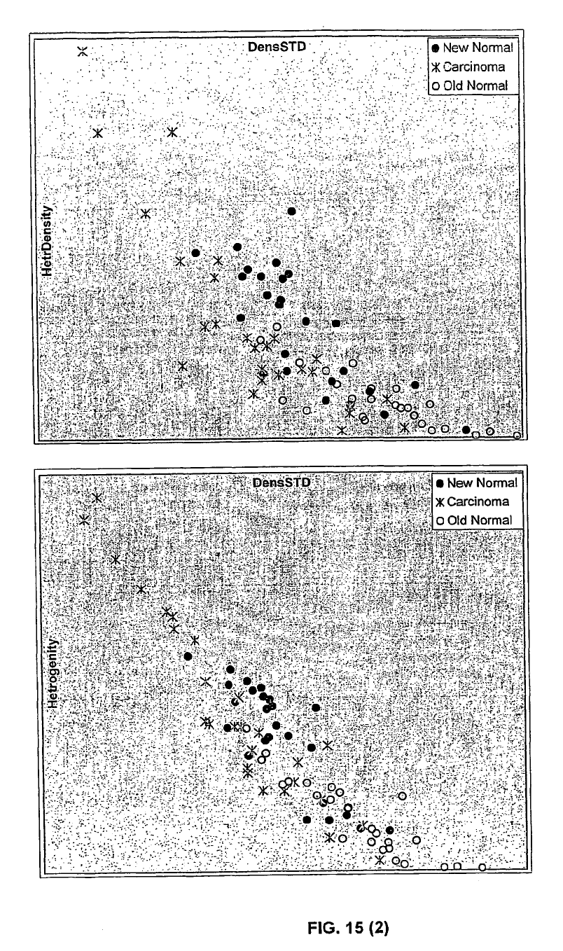

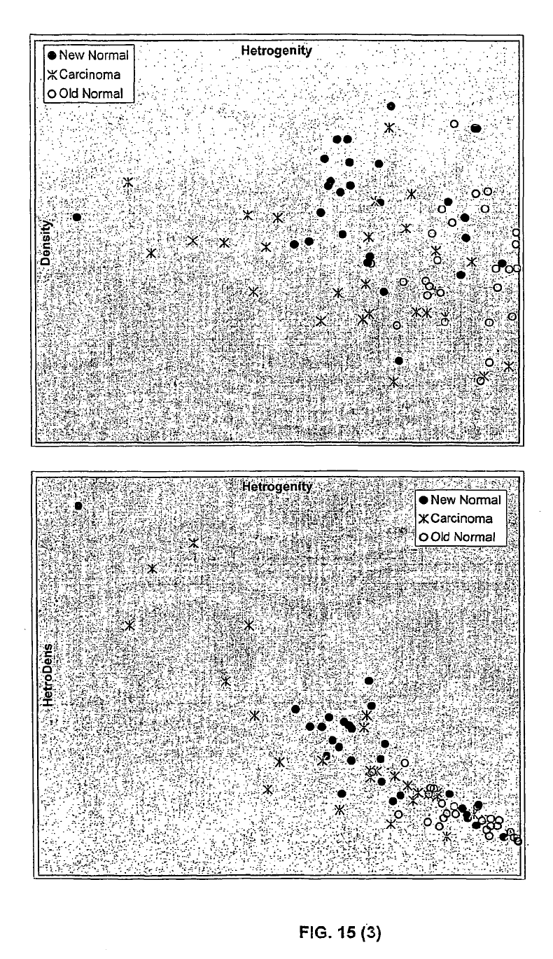

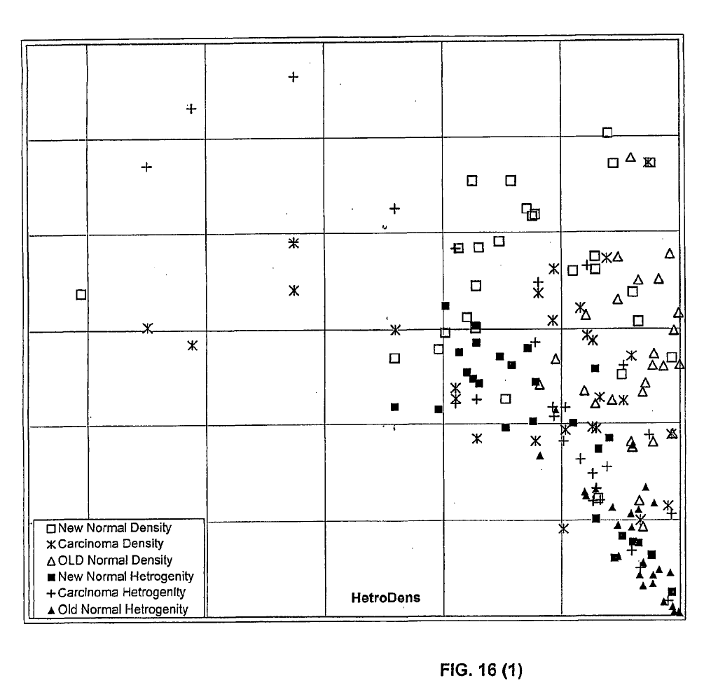

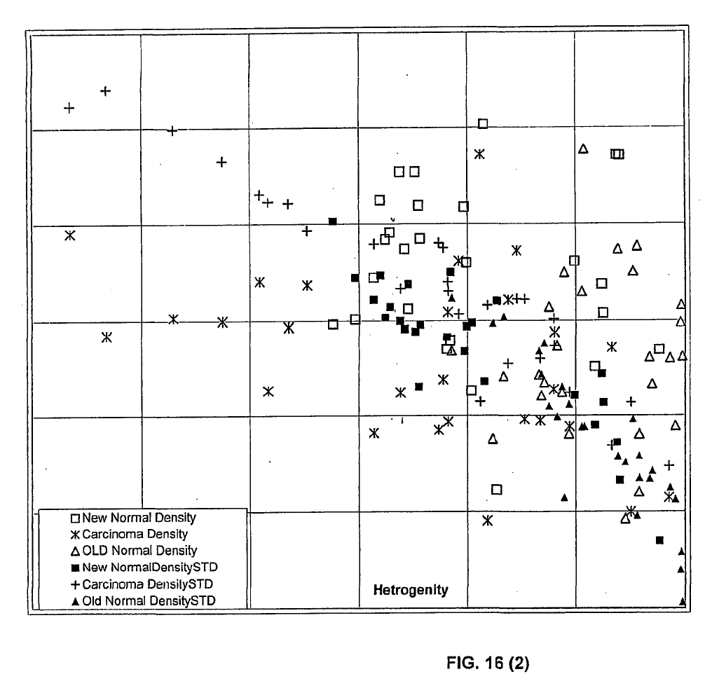

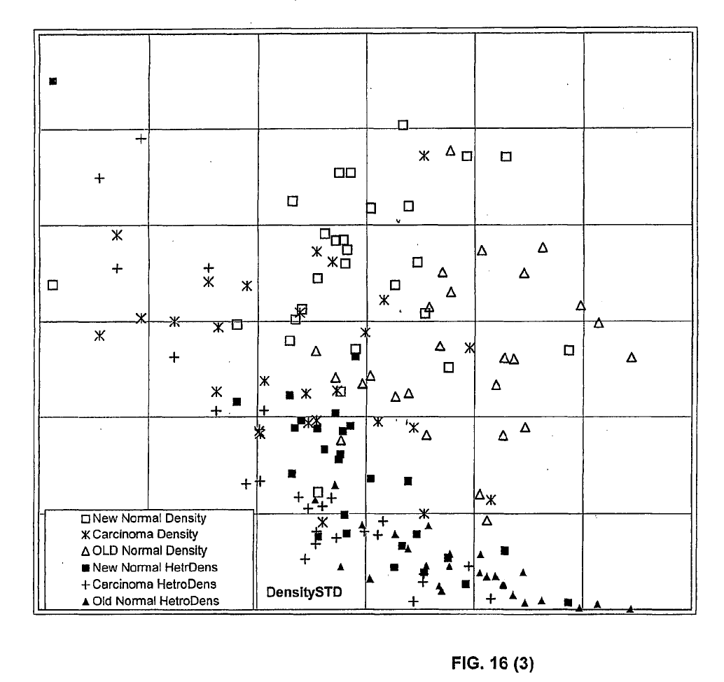

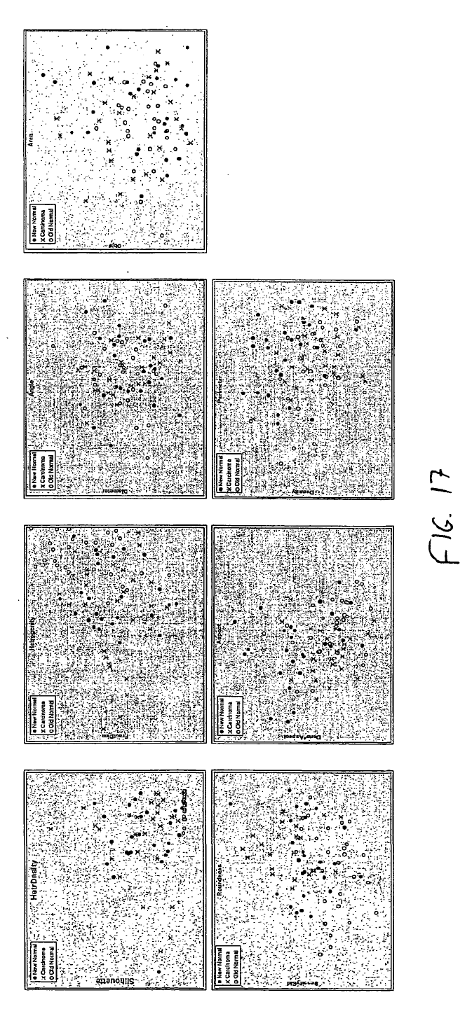

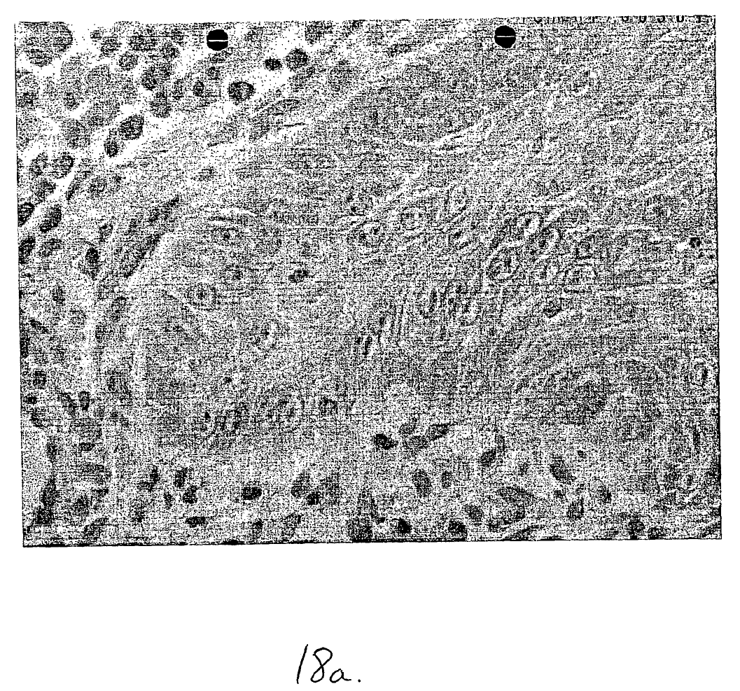

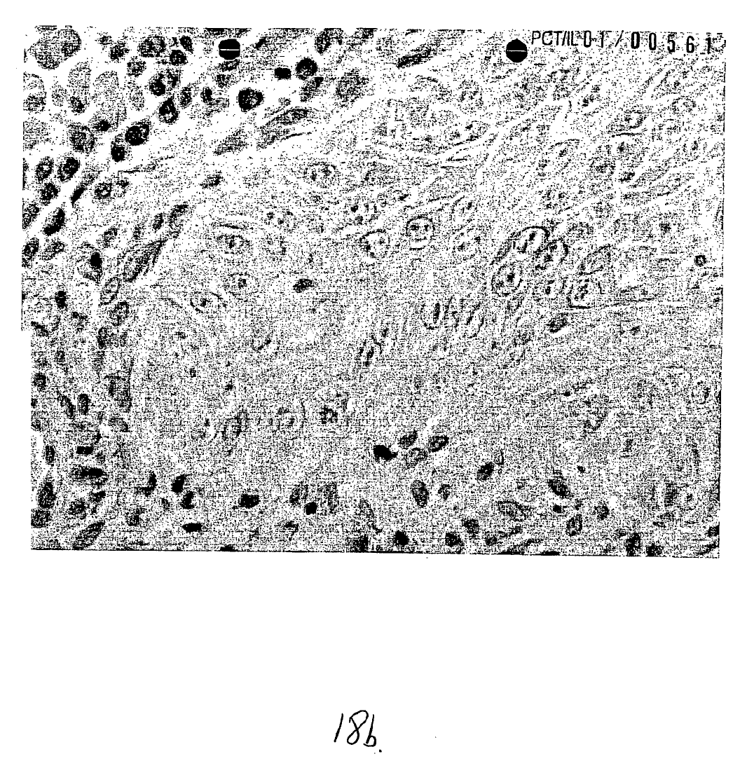

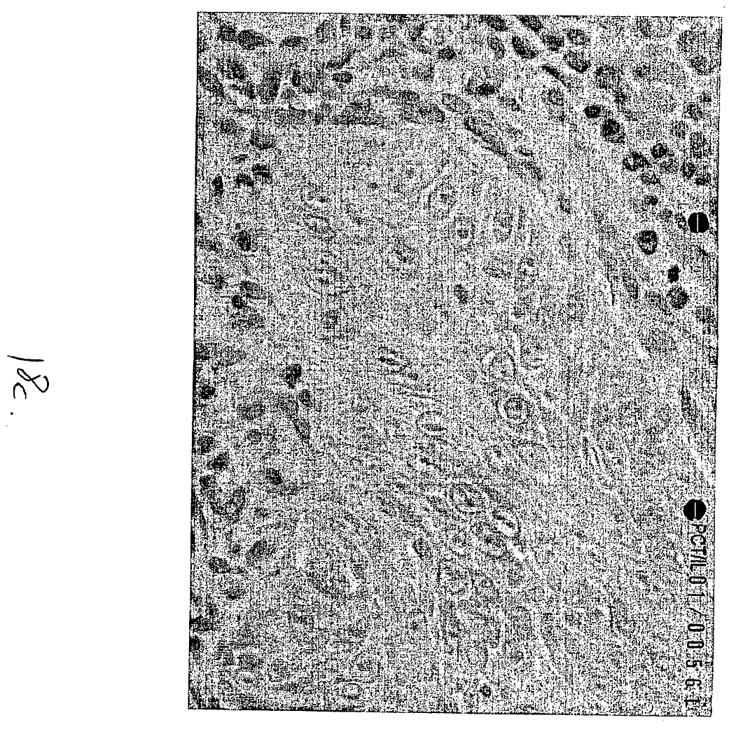

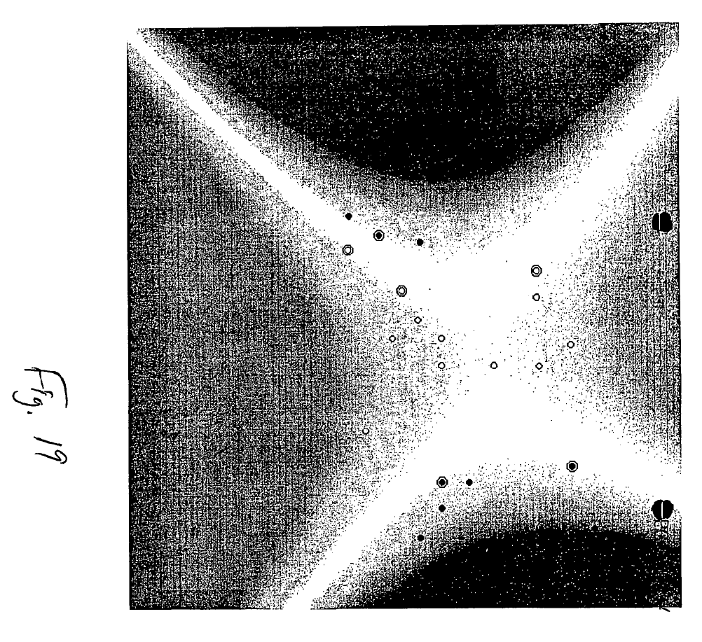

FIG. 5 illustrates an example of a Web Site related to the system. The Web Site provides pathology information, system support and pathology consultations. The pathology consultation scheme is illustrated in FIG. 6 and comprises selecting an advisor, sending data and pay, and obtaining the appropriate results.

In the exemplary embodiment of the invention, the Web Site comprises a remotely accessible database, which includes quantitative data extracted from the images by the learning mode and a searching kernel for the tissue type of the requested histological exam. Access to the web site enables analysis of new samples by using the search kernel and Al (artificial intelligence) software to compare features locally extracted to features presented by database.

Image Acquisition Stage

In accordance with FIGS. 3 and 4, the learning mode software component of the invention determines what data—images, parameters, and other quantitative data—are required or desired for a complete database in order to construct a search kernel. The same software controls the operation of the local diagnostic system. While carrying out the invention, image analysis software enables either an intelligent scan of the specimens to locate region of interest where the significance of the data is higher or aid the pathologist in his manual navigation within the image. By means of the capturing devices, at least six images are acquired: one, a general view, at 2×-6× magnification, is used to examine the macroscopic specification of the specimen in order to locate the areas of interest. Two images—medium magnification 5×-20×, of the boundary area between the normal tissue and the suspected lesion in various depths, two more of the suspected lesion itself, and one image in high magnification 20×-100×, used as reference for calibrating the system. Then the images undergo a standardization procedure controlled by analysis software component.

The first stage of the automatic image standardization procedure comprises color and light correction, calibration and thresholding of color lighting etc. for comparative purposes of tissue, cellular and nuclear behavior. Standard, off-the-shelf image enhancement software may be used at this stage. In the next stage, the invention first compares the range of features in the normal-tissue image to a search kernel, which comprises an updated standard determined by the system, and then sets accordingly the thresholds of the features extracted by the system for the diagnosis of expected malignancy. The “individualization” algorithm hereby addresses the natural variance in the normal population, facilitating higher sensitivity in detecting malignancy overcoming. Once, a salient image is acquired, and the image standardization algorithms are carried out, the image is analyzed in depth by computer vision and/or imaging programs that perform measurements according to the desired features which make up the vector for the tissue type, and provide values for the vector of features derived from the image in accordance with the kernel.

The individualization program essentially compares the readings for the features of the vector for both the normal tissue sample and the suspected tissue sample of the particular patient against Gaussian curves for each of those features. Once it is determined where along the “normal” curves the patient's readings fall, all of the data set for that patient are adjusted accordingly. For example, if it is seen that both the normal and suspect tissue samples of the patient appear to lie at the low end of the curves for a particular feature (e.g. nuclear density), then all the data which correlates with that feature will be adjusted accordingly, practically in a point-to-point manner.

An alternative embodiment of the invention comprises a smart scanning procedure that detects the boundary layer and automatically acquires images along it. This alternative enables the pathologist to get a general knowledge about the case, before beginning his diagnosis.

Analysis Stage

The desired feature vector is classified using advanced pattern recognition methodologies supported by support-vector machine (SVM) technology or neural network or fuzzy logic or similar Al methodology. A feature vector is the entire class of morphologic and/or pathologic features which are measurable in a histological sample. A desired feature vector may be a somewhat limited subset of features taken from a feature vector, for example depending on age range of the patient, race or some other macro demographic or biological group factor. A desired feature vector forms a master set of features from which specific features are selected for defining a search kernel. Regarding the learning-mode, these methodologies enable the training of classifiers for recognition of patterns based on offline learning from data classified normally by expert. (see “An Overview of Statistical Learning Theory” by V. N. Vapnik).

Supervisory software which supervises the software components of the invention then constructs a data-package file comprising the normalized and individualized raw features extracted from the images, and other details such as specimen properties, macro description (general observations such as landmark locations in low magnification image), date, patient details etc. The file is stored in a database with a unique I.D. Then the file is either manually analyzed by one or more pathology consultants and/or sent for computer automated pathological analysis (CAPA) performed per the search kernel. The analysis software guided by the search kernel controls the comparison of the data, relative to a specific patient, to the data comprising the database. The comparison results in a diagnostic conclusion in at least one of the following ways, or a combination thereof. Where the diagnostic process occurs in an automatic manner, the analysis software may completely control the formulation of diagnostic conclusions carried out according to the search kernel. The results that appear are preferably not a binary decision but a graded scoring that measures the “probability” or the degree of malignancy (optionally implemented by neural network (NN) or Fuzzy Logic). Alternatively, although diagnostic conclusions can be drawn automatically, analysis of the comparison of data may be carried out in an interactive manner with pathology experts. It is also possible, in difficult diagnostic situations to have both the analysis software and experts work in parallel and interactively.

An object of the present invention is interpreting heuristic/subjective information used by the pathologist and converting it to quantitative data, which can be used in the partially or totally computerized diagnosis. As mentioned above, the search kernel carries out the main part of the local analysis by comparing features of acquired images to features comprising the database. The determination of what features are required for the search kernel is made by Interpreting heuristic information typically used by a pathologist and converting it to quantitative parameters.

Tables 1 and 2 illustrate the raw data for features extracted by an analysis procedure based on a search kernel built for diagnosis of carcinoma.

The conversion process is carried out as follows:

1) Definition of a top- pathologist's heuristic conclusion from an image by a number of specified features and complex features (where possible); and

2) Reduction of said features to mathematical formulas.

Comparison is then performed, by algorithms based on the mathematical formulas, of numerical data obtained locally, i.e. from the specific sample in question, with the data comprising the systems database and refinement of the conclusions using artificial intelligence tools (SVM).

Complex features comprise combinations of morphological and/or pathological features which are shown, by SVM or by observation using the software of the present invention, to somehow be linked in occurrence with respect to particular types of malignancies. Complex features are shown hereinbelow to be particularly accurate in distinguishing between malignant and normal tissue. Complex features include: Heterogeneity_Density, Silhouette, Heterogeneity, Fractal Dimension, Density Aspect, Density Std (Standard Deviation). Another complex feature may be the combination of Angle and Aspect Ratio. Heterogeneity refers to gray levels inside nucleus. Density means nuclear density. Silhouette means combination of Aspect Ratio and Roundness. Fractal Dimension refers to shape of nucleus. Density aspect refers to variation in nuclear gray-levels.

More particularly, the approach of the present invention is to use the prior knowledge of the pathologist and to convert that to mathematical algorithms. It is different from known neural networking as it provides greater control on the criteria used and on their priority. To sharpen the dialogue between the pathologist and the algorithm, 4 levels of binarization may be used to characterize the image. The pathologist's subjective description of the histological sample is found in these 4 levels.

The following is an example of a technique developed to set these 4 ranges in the most accurate way. Two magnifications are used in the automated process −4× for initial determination of the various layers in the tissue. As we are discussing first the carcinoma of the epithelial tissues then the layers are: basal cells, spinous cells, keratin cells from the normal internal tissue to the skin boundary respectively. Once the various layers are determined higher magnification, 40×, images are acquired and analyzed using the vector of features.

An algorithm is applied to pre-sort the acquired images and determine whether they can be used for automatic classification or the user should be prompted to acquire improved images. Pre-sorting assures that low quality images with inaccurate focus, saturation, poor staining etc. will not degrade the decision process.

The binarization technique effects a representation of the various layers of a histological sample, among them are background, nucleus and cytoplasm.

As the inspection is histological, several textural criteria are used to classify the tissue, among these are fractal, intensity variance, nuclear density, heterogeneity, area, aspect, cellular density, standard density, perimeter, roundness, diameter at an angle, diameter in silhouette, aspect density, heterogenity density, etc. These are also useful in combination with a textural algorithm for orientation determination. This orientation determination algorithm is based on orientation of a cell and its nearest neighbor. It reflects the subjective appearance of “flow” seen on tissues.

Observations on tissues have indicated that there is a large variation between normal tissues of different people. This fact blurs the classification between normal and malignant tissues. To overcome this problem the present invention determines first the range of features in a specific normal tissue and accordingly sets the range of features for expected malignancy. This “individualization” algorithm facilitates higher sensitivity in detecting malignancy by accounting for the natural variance in the normal population.

Out of the possible features vectors available, the present invention uses different sub-sets for the different zones. In each zone we apply the sharpening procedure compared to the statistics of a normal specific tissue.

In general, as was discussed hereinabove, it is advantageous to limit the number of features used for the actual decision and to use the features that indeed represent the mode of decision of the pathologist. Further the complexity of the process of inspection increases according to n cubed where n is the number of features. As such different features are used for different zones and also we have made a special effort to define sets of features as one feature which has better correlation to human observation. For example the multiplication of ARXSF better represent elongated objects with fuzzy edges. We have defined 8 such complex features and they better differentiate between normal and malignant tissues. With reference to our results of conventional features compared to the 8 complex features, the superiority of the complex features is clear.

It is worth while to elaborate here and to explain that the incentive is to develop an easy to use cost effective system. To take hundreds of features with no prioritization raises the system cost and complexity without really improving the accuracy compared to the present invention wherein, as much as possible, the system benefits from procedures, experience and methodologies of top pathologists.

The decision process itself is based on a SVM algorithm which is compatible with the classification rational outlined above, taking full advantage of the pathologist experience.

The present invention uses composite features, such as cell silhouette, perimeter at an angle, diameter at an angle. The composite features are optimized per layer.

As an example, with reference to FIGS. 9a and 9b, there is shown two images, one normal and the second malignant, respectively. Measurement is automatically done to obtain the mean and standard deviation of a conventional feature—the Aspect Ratio, versus the mean and standard deviation using one of the composite features—the silhouette. This is done for both the normal tissue and the malignant tissue. The results appear below:

| | |
|---|---|
| Mean | Standard deviation (Std) |

Normal Tissue Sample

| | | |
|---|---|---|
| Aspect | 2.49 | 1.17 |
| Ratio | | |
| (AR) | | |

Carcinoma Tissue Sample

| | | |
|---|---|---|
| Aspect | 2.20 | 1.27 |
| Ratio | | |
| (AR) | | |
| Silhouette | 137 | 85.5 |

It is apparent that the mean shows emphasized difference between normal and malignant tissues when one compares the mean or Standard Deviation of AR for

Normal versus the mean for AR of Carcinoma. Similarly, comparison of the composite feature Silhouette shows emphasized differences whether one looks at mean or Standard Deviation. A combination of composite features thus also lowers the Std and facilitating a high percentage of correct classification.

In addition the Al tools can compensate for heuristic decision factors, which can't be reduced to mathematical formulas but still, in some cases, have significant effect on the pathologists decision (the “gut feeling” factor). One preferred Al technique which may be used by the present invention, as has been previously mentioned, is the SVM (support vector machine). Thus, features, even those not in the search kernel, may be processed to by the SVM to try and detect subtle relationships which could affect how the search kernel appears in the future.

The Image Analysis procedure examines textural properties extracted from the acquired image such as granularity, density of the cell nuclei, fractal dimensions etc. Those measurement results can form a basis for the predicate calculations used by the search kernel and the Al techniques.

As mentioned above, another aspect of the present invention is the support of the pathologic inspection by telediagnosis and teleconferencing

The procedure of transmitting the images can introduce distortion and errors into the images and therefore the invention provides validation of the received images in order to insure that the images didn't suffer from significant distortion.

In addition, the present invention provides enabling the receiver of the images to decide what images should be included in the diagnosis procedure (manual or automatic procedure) for effective telediagnosis. This property is achieved by sending many images, each image sent together with registration data, wherein the receiver is able to navigate between the images and pick whatever images he wants.

An exemplary embodiment of the invention comprises the construction of a special Internet portal, holding the database for comparative analysis and also providing options of

(1) Local acquisition of new information and updating search kernel accordingly;

(2) Acquisition of expansions of the system;

(3) Consultation with a group of pathology experts for the various types of cancer.

These experts operating in the portal use the special computerized diagnostic tools as part of their evaluation.

The system also may support upload and download of database image packets (i.e. the packets formed by the images and their raw data values) and search kernels from aforementioned Internet portal or mirror site databases. A user of a system according to this invention may enter the Web Site and to enlarge the capability of his own system by retrieving the systems kernel data arrays. Thus it is possible to add new analysis capabilities or new kernels for different kinds of cancer or to improve the performance of the diagnostic system.

In addition to the consultation between colleagues, as hereinbefore described, a Web Site facilitates consultation with a number of known pathologists, who reply to the questions posted to the Web Site, or discuss and suggest solutions to urgent problems which are brought up using advanced real time consultation facilities.

Examples of hardware that is included in the local diagnostic means are personal computers, frame grabbers, color TV cameras, biological microscopes etc.

In addition to the aforementioned web site, several methods of telediagnosis can be used in carrying out the present invention. The diagnostic system can be on both sides of the communication link or only on the transmitting side or only on the receiving side. The first method allows implementing the diagnosis routine on both ends (using image-capturing techniques) wherein the database is significantly enlarged taking advantage of the cooperation between the two sources communicating. In the second method, automatic diagnostic results and images are transferred, for further diagnosis. In the third the diagnostic system and the expert are in the receiving side and the transmitting side just provides the relevant images by means of image capture and transfer (according to image capture provisions).

In another embodiment of the present invention the transmitting side provides a file comprising figures describing the entire suspected tissue, permitting choice of images by the receiver for re-running an auto analysis or performing one manually. In order to enable navigation through the received images, the images are sent together with registration data, which arranges all the images into a “big image” insuring the regional relationships between the sent images are right.

This offers a cost-effective solution for automatic pathologic diagnosis.

With reference to apparatus viewpoint, according to an embodiment of the invention, there are three methods of image capture and transfer:

1) The use of a 2D camera and a frame grabber that will interface at the required bandwidth to the communication channel;

2) The use of a digital 2D camera to interface to the computer memory from which the data is then transferred via the communication channel;

3) The use of a 1D camera and a scanning stage with high resolution to scan the image and construct a 2D image in the computer memory. This latter technique is especially advantageous and cost effective in acquiring high-resolution images. It is very useful also for other medical images such as X-ray and MRI.

New Drug Assessment and Monitoring

An additional embodiment of the present invention is a method for new drug assessment (NDA). The need to assess new drugs is growing with the progress made in biotechnology. Since the assessment should comply with the FDA regulation, careful supervision on the experiments is very important for the drug industry. One of the techniques used in NDA is the interaction of the drug with known cultures or germs, or verifying its interaction with living tissues. The technique known in the art, to inspect the interaction, is visual inspection of the samples and determination of the level of interaction. The present technique also sets the limit of concentration required for effective interaction. Whereas the inspection procedure is well defined, the verification of the test by the drug manufacturer is cumbersome and the ability for remote inspection is very limited.

The present invention's features of remote image normalization (standardization) and analysis capability and web interconnection, facilitate remote supervisions of new drug assessment trials. The present invention enables the application of a pre-defined inspection algorithm and later transmission of the analyzed data as well as the visual image to the drug company for inspection and archiving. The present embodiment allows standardization of experiments conducted around the world, and thus better tracking and faster cycle time in the evaluation of a new drug. By way of example the following description relates to antibiotics specifically. However this embodiment can be used for other applications where the need for central inspection using visual information exits.

Two methods are suggested for carrying out the present embodiment:

1) In the first method a microbiological culture of microbes colors a Petri dish and acts as a breeding ground. Six separate centers of nuclei of antibiotic matter, in different concentrations, are spread in the Petri dish. After an indicated period the Petri dish is observed visually. The areas in which the antibiotics have operated will look like a bright circle, free of microbes around each antibiotic center. The efficiency of the antibiotic matter is a function of the I.O.D. [NIR: STANDS FOR?] value in relation to the concentration of the matter in each center.

In accordance with the present invention, this first method comprises the following stages:

a) Taking a photograph of the Petri dish.

b) Correction of the picture and its normalization in accordance with the first picture that contains the background surface of the optical apparatus.

c) Finding the antibiotic centers and calculation of analyze barrier.

d) Full analysis, which will define the circles around the antibiotic centers and will calculate the I.O.D.

e) Showing results: a scheme of I.O.D. in dependency with the antibiotic concentration.

2) In the second method a microbiological culture of microbes colors the Petri dish and is used as a growth ground. In the center there is a stripe containing antibiotic matter in changing concentration. After incubation time microbe free surfaces will appear around the stripe. Antibiotic efficiency will therefore be according to the size of the clear surface in dependence with the concentration. In accordance with the present invention, this second method comprises the following stages:

a) Taking a photocopy of the Petri dish.

b) Correction of the picture and its normalization in accordance with the first picture that contains the background surface of the optical apparatus.

c) Building a concentration picture around the examined stripe and showing it as a picture in which height lines correspond the concentration

d) The result will be the measure of the concentration on the first place over the stripe around which the microbe free surface size will be higher than the minimum value.

It is apparent that the invention provides a new system for diagnosis and telediagnosis of histological tissues. There are various aspects to the invention, which are original as a whole but also individually. These aspects are: an established computerized hierarchy of the diagnosis process, a procedure for an automatic scanning routine along the boundary layer, an algorithm library for analyzing the tissue and determining the malignant cases, an Al algorithm that support the decision process as well as facilitates quantification of heuristic/subjective impressions used by the pathologist, means for image capture especially for stationary images and transfer via the Internet or conventional telephone lines, means for man-machine interaction on the remote unit that will simulate as much as possible the real interaction, and an Internet portal for consultation using the procedure suggested here as the base line. Other benefits of the present invention are the ability to archive and to track history.

Whereas the major aim of the present invention is in pathology, the image capture and scanning procedures are relevant and applicable to other areas where analysis of high-resolution images is required, a few examples are being X-ray and MRI imaging procedures.

1. A histological system including:
(1) A database, comprising images and quantitative information on diagnostically relevant features.
(2) An inspection procedure for the examination of a suspected object.
(3) Analysis tools for comparison of the locally obtained data to those stored in the database.

2. A system according to claim 1, wherein the diagnostically relevant features are textural, morphometric, calorimetric and densitometric features.

3. A system according to claim 1, wherein the database is based on a structure and program that are designed so as to provide the database completeness, accessibility, and as far as possible, universal value.

4. A system according to claim 1, wherein the local acquisition of data is based on a structure and program which correspond to those of the database, so as to facilitate comparison, feature v. feature and/or complex features v. complex features, of the data locally obtained and representing a specific object, to those stored in the database.

5. A system according to claim 1, comprising an automatic image standardization procedure.

6. A system according to claim 5, wherein the automatic image standardization comprises color and light correction, calibration and thresholding for comparative purposes of cellular and nuclear structure and determination of areas of interest.

7. A system according to claim 1, comprising capturing images from both normal and suspected tissues for normalization of the features of specific image according to systems threshold, and extracting object's and textural features.

8. A system according to claim 1, which includes transferring images of relevant tissues over the Internet for examinations by experts at a location different from that at which the diagnosis is to be carried out.

9. A system according to claim 1, which comprises, as components, microscopes, computers, image capturing devices and telecommunications means.

10. A system according to claim 9, where the components are supplemented, and their operation is improved and controlled, by Al-based diagnosis software.

11. A system according to claim 1, further comprising a procedure for searching the suspect tissue along the two sides of the boundary layer using a motorized stage. Add registration

12. A system according to claim 11, comprising an interactive method for transferring commands to move said motorized stage on a remote microscope.

13. A system according to claim 11, comprising initial scanning of the tissue according to a defined routine, performing initial analysis and thus giving the expert an initial indication on the examined tissue.

14. A system according to claim 1 designed particularly for the diagnosis of cancerous growths.

15. A system according to claim 14, further comprising analyzing the data acquired and obtaining guidance to probability of cancerous tissue and/or to the need for more information.

16. A system according to claim 1, comprising:
(1) An Al software for controlling the acquisition and storing of data for the database.
(2) An Al software for the operation of a local diagnostic means, means for transmitting said local diagnostic data to an Internet Web Site, means for comparing said local data with those comprised in the database and means for reaching from said comparison a diagnostic conclusion.

17. A diagnostic system according to claim 16, wherein the diagnostic conclusion is reached in an automatic manner.

18. A diagnostic system according to claim 16, wherein the diagnostic conclusion is reached with the intervention of experts.

19. A diagnostic system according to claim 17, wherein the Al software defines what images, parameters, and other quantitative data are required or desired for a complete database.

20. A diagnostic system, substantially as described and illustrated.

21. A system and apparatus for new drug assessment (NDA) comprising:
1) Visual examination of an experiment.
2) Correction of the picture and its normalization.
3) Communication means for transmission of said data to any preferred location.

22. A system and apparatus for remote supervision of NDA experiment.

23. A predefined NDA inspection algorithm

24. A system and apparatus for transmission of normalized visual data to a different location for further analysis.

25. A system and apparatus for transmission of visual data to a different location for normalization and further analysis.

26. A system and apparatus for transmission of normalized and analyzed visual data to a different location for inspection and/or archiving.
